(19) United States
(12) Patent Application Publication
(10) Pub. No.: US 2004/0096857 A1
(43) Pub. Date: May 20, 2004

(54) Multiple-inspection multiplexing method and suspension for multiple-inspection multiplexing

(76) Inventors: Masayuki Machida, Ibaraki (JP); Hiroko Hagiwara, Ibaraki (JP); Hideji Tajima, Chiba (JP)

Correspondence Address: Warren B Kice, Haynes & Boone, Suite 3100, 3100 Main Street, Dallas, TX, 75202, US

(21) Appl. No.: 10/450,584

(22) PCT Filed: Dec. 14, 2001
(86) PCT No.: PCT/JP01/10994

(30) Foreign Application Priority Data
Dec. 15, 2000 (JP) 2000-382783

(51) Int. Cl. C12Q 1/68; G06F 19/00; G01N 33/48; G01N 33/50

(52) U.S. Cl. 435/6; 702/20

(57) ABSTRACT

In relation to a multiple inspection multiplexing method and a suspension for multiple inspection multiplexing, it is an object to provide multiple inspection multiplexing method and a suspension for multiple inspection multiplexing which by performing inspections of multiple types together in parallel, processing can be performed efficiently both timewise and spacewise, and since the same conditions can be set for inspections of each type, inspections of high reliability can be conducted.

A suspension contains; a detection substance group for multiple inspection types labeled so as to be identifiable among respective inspection types, and a fine particle group for the multiple inspection types having for each of the inspection types bonding substance groups for multiple inspection types selected according to the inspection description of the inspection types based on whether they bond with or do not bond with the labeled detection elements for each inspection type, and inspection of multiple inspection types is performed in parallel using the suspension by detecting for each of the inspection types whether the labeled detection elements are carried by fine particles or not or to what extent they are carried.

### TECHNICAL FIELD

The present invention relates to a multiple inspection multiplexing method, and a suspension for multiple inspection multiplexing. Particularly, the present invention relates to all manner of fields, such as fields requiring inspections, analyses and diagnoses relating to genes, the immune system, proteins, amino acids, and biopolymer such as sugars, for example engineering fields, agricultural fields such as food, produce and seafood processing, pharmacology fields, medical fields such as sanitation, health, immunization, disease and genetics, and scientific fields such as chemistry or biology.

The present invention relates particularly to a multiple inspection multiplexing method, and a suspension for multiple inspection multiplexing, suitable for use in the mutational analysis of genes, polymorphic analysis, mapping, base sequence analysis and expression analysis.

### BACKGROUND ART

Heretofore, in order to perform gene probe (a labeled specific DNA or RNA array) analysis, the targeted gene arrangement is amplified using a gene amplification technique (for example PCR), and then using a hybridization/ligation detecting method, the array of the probe hybridized with the target array is separated and made able to be inspected (for example, the probe contains a combination of magnetic particles and acridinium ester), thereby enabling determination of whether or not a specific array exists (Japanese laid-open publication No. Hei 9-510878).

In particular this method is a method of identifying a target polyacid array, and involves; (a) when probes of two types are ligated, making the probes complementary to part or all of the anticipated sequence of the target polyacid, and bonding one of the probes to a segment which enables it to be easily separated from the reaction compound, and bonding the other probe to the label, (b) mixing the probes with the target polyacid, to hybridize the probes with the target polyacid, (c) adding a ligation reagent, (d) transforming the reaction compound to thereby separate the probe from the target polyacid, (e) separating the probes using the aforementioned segment enabling easy separation, and (f) analyzing the separated probes to determine whether or not the label is bonded to the probe.

However, in the above gene probe analysis method, analysis is always limited to the inspection of one kind of base sequence. Consequently, in performing a plurality of analyses, the above method needs to be carried out for each type separately. As a result, this has such problems as; a need to prepare large quantities of many types of vessels, reactants and reagents for each type, a need to wash the vessels and the like for each inspection, a need to provide pyrostats and the like, and a need to perform processing for each type sequentially, which requires an extension of the processing time, and furthermore, requires a large processing space and processing facility.

Moreover, this has a problem in that because a variety of tasks need to be performed for each of the inspections of each type, processing is complicated, requiring a lot of time from the operator.

In addition, this has a problem in that preparation is necessary to ensure that cross-contamination does not occur between the types of suspensions and reagents, which differ for each process, and supervision of the processes therefore requires a great deal of time.

Consequently, the present invention aims to resolve the problems outlined above. A first object is to provide a multiple inspection multiplexing method and a suspension for multiple inspection multiplexing which by performing inspections of multiple types together in parallel, not only reduces the processing time but also requires a smaller work space, and enables processing to be performed efficiently.

A second object is to provide a multiple inspection multiplexing method and a suspension for multiple inspection multiplexing which by performing inspections of multiple types together in parallel, allows even minute amounts of a substance used for each inspection to be handled collectively in a bulk amount, and therefore is easier to handle and more convenient.

A third object is to provide a multiple inspection multiplexing method and a suspension for multiple inspection multiplexing which by performing inspections of multiple types together in parallel, enables the articles such as reagents which are required in common between inspections, and facilities related to environmental factors such as temperature, and manpower, to be conserved, thereby reducing the inspection costs.

A fourth object is to provide a multiple inspection multiplexing method and a suspension for multiple inspection multiplexing which by performing inspections of multiple types together in parallel, enables the same conditions to be set for inspections of each type so that comparison of inspection results between each type can be carried out under identical conditions, thereby enabling the discovery of essential differences between the types, and the acquisition of reliable and highly accurate inspection results.

A fifth object is to provide a multiple inspection multiplexing method and a suspension for multiple inspection multiplexing which is particularly suited to performing inspections and processing which require repetition of a large number of simple processes, due to the need to obtain a large volume of information such as with determinations of base sequences of genetic material, for example.

### DISCLOSURE OF THE INVENTION

In order to resolve the above problems, a first aspect of the invention is a method of multiple inspection multiplexing for conducting in parallel inspections of multiple inspection types comprising:

a generation step for generating labeled detection element groups for multiple inspection types labeled so as to be identifiable among respective inspection types;

a processing step in which at least a generated labeled detection element group for the multiple inspection types, and a fine particle group for the multiple inspection types having for each of the inspection types bonding substances for multiple inspection types selected according to the inspection description of the inspection types based on whether they bond with or do not bond with the labeled detection elements for each inspection type, are suspended in a liquid and processed, and

a detection step for detecting for each of the inspection types whether the labeled detection elements are carried by the fine particles or not or to what extent they are carried.

In this description “inspection types” refers to the multiple types of inspection to be performed in parallel. Inspection types can be classified according to the type of the target of the inspection, or the type of the inspection site in the same target of inspection, for example.

Furthermore, “labeled detection element” is a detection element which has undergone labeling, and “detection element” is a minute solid used in detection, of which large quantities are included in the suspension liquid for each inspection type. “Labeling” is performed by bonding with a labeling substance, for example. Labeling substances are not limited to optical substances, and labeling may be performed by various substances having instantaneously quantifiable other physical and chemical quantities. The “extent of carriage” refers to quantity of the carried amount, that is the extent of labeling during detection, and when labeling is performed optically refers the intensity of the light, for example. The aspect of “having bonding substances” includes cases in which the bonding substance is bonded, the bonding substance is adhered or fixed, the bonding substance is adsorbed, or the bonding substance coats the surface of the fine particles, or cases having the bonding substance bonded through the medium of another substance.

According to the first aspect of the invention, by detecting or not detecting the labeling condition for a given particle according to the labeled detection element carried via the bonding substance, the inspection type to be identified is specified from the label, and various information can therefore be easily obtained for the inspection type.

Furthermore, according to the first aspect of the invention, by multiplexing inspections for a plurality of inspection types, the inspections can be executed in parallel. Consequently, the processing time can be reduced, and efficiency can be improved, while also reducing the workspace required for the processing, and also allowing the use of more compact apparatuses for processing.

Furthermore, even with minute amounts used for each inspection, these can be handled collectively in a bulk amount, and therefore are easier to handle and more convenient. In addition, articles such as reagents which are required in common between inspections, facilities related to environmental factors such as temperature, and manpower, can be conserved, thereby reducing the inspection costs.

Moreover, the same conditions can be set for inspections of each type so that comparison of inspection results between each type can carried out under identical conditions, thereby enabling the discovery of essential differences between the types, and the acquisition of reliable and highly accurate inspection results. Furthermore, the invention is suited to efficiently performing inspections and processing which require repetition of a large number of simple processes, due to the need to obtain a large volume of information, such as with determinations of base sequences of genetic material, for example.

A second aspect of the invention is a method of multiple inspection multiplexing according to the first aspect of the invention, wherein the generating step comprises a step for suspending and bonding in a liquid for each of the inspection types, multiple detection elements, and labeling substances for the inspection types with predetermined types included at a predetermined molar ratio, and the type or the molar ratio thereof are made different so as to be mutually identifiable for each of the inspection types. Here for the “labeling substance” there is for example substances of, luminescent material such as fluorescent substances, substances which emit electromagnetic waves, substances which emit a magnetic field, substance having an electric charge, and so on.

According to the second aspect of the invention, labeling is performed by making labeling substances with a predetermined type included at a predetermined molar ratio different, so as to be identifiable between the respective inspection types. Consequently, in addition to the aforementioned effects, the effect is demonstrated of enabling identification between multiple inspection types (several hundred, several thousand or more than several tens of thousands, for example) using only a few types of labeling substances. Furthermore, accurate and precise labeling can be realized simply and within a statistical margin of error, by suspending multiple related substances for each inspection type.

A third aspect of the invention is a method of multiple inspection multiplexing according to the second aspect of the invention, wherein the generating process comprises a step for distributing all of the labeling substances for each of the inspection types to approximately all of the labeled detection elements for each of the inspection types, and bonding a single labeled detection element with only a labeling substance of one type.

According to the third aspect of the invention, in addition to the aforementioned effects, each detection element is bonded with only a type of labeling substance of one type. Consequently, the molar ratio equates to the detected strength of the labeling substances bonded specifically to the individual particles, and it is therefore possible to easily detect not only the presence or absence of the labeling substance but also the degree of such presence.

A fourth aspect of the invention is a method of multiple inspection multiplexing according to any one of the first through third aspects of the invention, wherein when inspecting for the suitability of the structure of an inspection target of the multiple inspection types, in the generating step the labeled detection elements are obtained by labeling the respective inspection targets, and in the inspecting step, the bonding substances are substances which are only bonded when the inspection target has a predetermined structure.

Here, “inspection target” includes biopolymers of for example; genetic material, the immune system, proteins, amino acids and sugars.

According to the fourth aspect of the invention, in addition to the above effects, it is possible to determine with high reliability and high accuracy, for example, the structure of a DNA base sequence or the like.

A fifth aspect of the invention is a method of multiple inspection multiplexing according to any one of the first through third aspects of the invention, wherein when conducting an inspection to determine an unknown structure of an inspection target for each of the inspection types, in the generating step the labeled detection elements are known structure elements of multiple types expected to bond with the bonding substances, only when the unknown structure is present, and are labeled so as to be mutually different, and the unknown structure is determined from the known structure elements bonded to the bonding substance.

According to the fifth aspect of the invention, in addition to the aforementioned effects, unknown structures of the inspection target substance for the multiple inspection types can also be determined with high accuracy.

A sixth aspect of the invention is a method of multiple inspection multiplexing according to any one of the first through third aspects of the invention, wherein when inspecting for the presence and degree of presence of the inspection target, the detection elements and bonding substances in the generating step and the bonding step are substances selected so as to mutually bond only when the inspection target is present, and in the processing step, the inspection target is also suspended.

According to the sixth aspect of the invention, in addition to the above effects, the invention can be easily applied to inspections relating for example to whether or not a microbial species is present in a given sample. Accordingly, inspections for the presence of colon bacillus, O-157, or the like can be executed easily and accurately.

A seventh aspect of the invention is a method of multiple inspection multiplexing according to any one of the first through third aspects of the invention, wherein when inspecting for the suitability of a structure of a predetermined inspection site of multiple inspection types of genetic material, in the generating step genetic material, such as a DNA fragment, cleaved so that each single chain inspection site includes a single inspection type, and labeled, is generated as the labeled detection element, and the bonding substances for the inspection types are genetic material having a single chain base sequence selected so as to bond or not to bond with the inspection sites of the genetic material, if the structure is normal or abnormal.

According to the seventh aspect of the invention, in addition to the above effects, material cleaved so as to include a single strand inspection site on which inspection for the suitability of the structure of a single type of DNA or the like is performed, is used as the labeled detection element. Accordingly, it is possible to specify efficiently and accurately a plurality of mutations in the DNA base sequence, in parallel.

An eighth aspect of the invention is a method of multiple inspection multiplexing according to the fourth aspect of the invention, wherein the generating step comprises; an amplification step for amplifying a double strand DNA fragment by the PCR method by mixing: double strand DNA having inspection sites for multiple inspection types; a primer group labeled for each of the inspection types with a labeling substance bonded with one end, into which is inserted a recognition site of a type IIS restriction enzyme which has a cleavage site in which an inspection site downstream of a 3′ end of the primer acts as a cohesive end; and a primer group paired with this primer group, and then amplifiing double strand DNA fragments by the PCR method; and an enzyme reaction step for processing the amplified DNA fragments by a type IIS restriction enzyme, to generate as the labeled detection element, DNA fragments having the cohesive end of the inspection site at the other end, and the processing step, for each inspection type, suspends in a liquid; the labeled detection element, and a fine particle group of multiple inspection types having a DNA fragment which has a cohesive end having a base sequence capable of bonding when a base sequence of the cohesive end of the labeled detection element is normal, and mixes these, to perform a ligation reaction.

According to the eighth aspect of the invention, a DNA fragment with one end labeled and the other end having the cohesive end of an inspection site is used as the detection element, this DNA fragment being obtained by providing upstream of a 3′ end, a type IIS restriction enzyme array with a double strand DNA having multiple inspection sites, in which the recognition sites and the cleavage sites are separated by at least 10 base pairs for each inspection site, and processing this DNA by a type IIS restriction enzyme using labeled primers for multiple inspection types, and primers for multiple inspection types paired with these.

Accordingly, with the eighth aspect of the invention, in addition to the aforementioned effects, by using a type IIS restriction enzyme recognition sequence, it is possible to cleave the double strand DNA at any position without adversely affecting the inspection sites, which allows inspections which are diverse or versatile to be performed.

A ninth aspect of the invention is a method of multiple inspection multiplexing according to the sixth aspect of the invention, wherein the generating step comprises an amplification step for suspending in a DNA extract in which multiple unknown DNA is suspended: a primer group labeled to as to be identifiable for each inspection type, and which for each of the inspection types begins DNA synthesis for known multiple inspection types which are the object of inspections; and fine particles which have many primer groups for multiple inspection types paired with the primer group, and amplifying by the PCR method.

According to the ninth aspect of the invention, in addition to the aforementioned effects, inspections to determine whether or not certain genetic material exists in a DNA extract in which unknown DNA which is the detection target of the multiple inspection types is suspended, can be preformed in parallel, and quickly and efficiently.

A tenth aspect of the invention is a method of multiple inspection multiplexing according to the fifth aspect of the invention, wherein when inspecting to determine the base sequence of genetic material which has mutation sites where mutations are predicted in each inspection type, in the amplification step,

as respective structure elements of the labeled detection elements there are primers having a base or a base sequence which is predicted to be mutated or inserted or not having a corresponding base or base sequence, in a position corresponding to the mutation site which is at the primer 3′end or the vicinity thereof, and structure different from this are labeled so as to be mutually identifiable,

and regarding the bonding substance there are fine particles having a large number of primers having a base or a base sequence which is predicted to be mutated or inserted or not having a corresponding base or base sequence, in a position corresponding to the mutation site which is at the 3′end of the primer or the vicinity thereof or separated upstream, and these are suspended in a DNA extract in which multiple unknown DNA is suspended and amplified by the PCR method.

Here “primer 3′end or the vicinity thereof” is because if separated from the 3′end of the primer, then the more separated, the base or the base sequence in the position corresponding to the mutation site is synthesized by the PCR method, so that the influence on the amplification is reduced, and there is the possibility of synthesis and amplification irrespective of differences in the base or the base sequence. Furthermore, “not having a corresponding base or base sequence” refers to a primer when a deficiency exists in the position corresponding to the DNA which is the inspection target.

According to the tenth aspect of the invention, in addition to the aforementioned effects, the known structure of the inspection target substance of the multiple inspection types can be determined with a higher accuracy.

An eleventh aspect of the invention is a method of multiple inspection multiplexing according to any one of the first through fifth aspects of the invention, wherein when inspecting the suitability of the structure for a predetermined inspection site for protein of multiple inspection types having predetermined immobilization sites, as to whether or not this exists or the degree of existence, the labeled detection element in the generation step and in the process step is a protein of multiple inspection types, and is labeled so that this is mutually identifiable for each of the multiple inspection types, by the labeling substance via a substance which is selected so as to specifically bond or not bond with the inspection site, and the bonding substance is a substance selected so as to specifically bond with the immobilization site.

According to the eleventh aspect of the invention in addition to the above mentioned effect, for the labeled detection element there is one which is labeled with a labeling substance via an antibody group selected so as to bond or not bond with the inspection site, so as to perform inspection of the suitability of the structure for protein of one kind, as to as to whether or not this exists or the degree of existence. As a result, for the inspection site of the protein of multiple types, inspection for mutant forms of the protein can be performed in parallel, and quickly and efficiently.

A twelfth aspect of the invention is a suspension for multiple inspection multiplexing containing; labeled detection element groups for multiple inspection types labeled so as to be identifiable among respective inspection types, and a fine particle group for the multiple inspection types having for each of the inspection types bonding substances for multiple inspection types selected according to the inspection description of the inspection types based on whether they bond with or do not bond with the labeled detection elements for each inspection type, and inspection of multiple inspection types is performed in parallel using the suspension by detecting for each of the inspection types whether the labeled detection elements are carried by the fine particles or not or to what extent they are carried.

According to the twelfth aspect of the invention, a similar effect to that described for the first aspect is demonstrated.

A thirteenth aspect of the invention is a suspension for multiple inspection multiplexing according to the twelfth aspect of the invention, wherein all of the labeling substances for each of the inspection types which are labeled by only bonding the labeled detection elements for the respective inspection types with labeling substances of respective inspection types, are substances which contain predetermined types in predetermined molar ratios, and these types or the molar ratios thereof are made different so as to be mutually identifiable for each of the inspection types.

According to the thirteenth aspect of the invention, a similar effect to that described for the second aspect is demonstrated.

A fourteenth aspect of the invention is a suspension for multiple inspection multiplexing according to the thirteenth aspect of the invention, wherein all of the labeling substances for each of the inspection types are distributed to approximately all of the labeled detection elements for each of the inspection types, and a single labeled detection element is bonded with only a labeling substance of one type.

According to the fourteenth aspect of the invention, a similar effect to that described for the third aspect is demonstrated.

A fifteenth aspect of the invention is a suspension for multiple inspection multiplexing according to any one of the twelfth through fourteenth aspects of the invention, wherein when inspecting for the suitability of the structure of an inspection target of the multiple inspection types, the labeled detection element groups are the inspection target groups labeled so as to be different for each inspection type thereof, and the bonding substance groups are substances which are only bonded when the labeled inspection target thereof has a predetermined structure.

According to the fifteenth aspect of the invention, a similar effect to that described for the fourth aspect is demonstrated.

A sixteenth aspect of the invention is a suspension for multiple inspection multiplexing according to any one of the twelfth through fourteenth aspects of the invention, wherein when conducting an inspection to determine an unknown structure of an inspection target for each of the inspection types, the labeled detection elements are known structure elements of multiple types labeled so as to be mutually different and expected to bond with the bonding substances, only when the unknown structure is present, and the unknown structure is determined from the known structure elements bonded to the bonding substance.

According to the sixteenth aspect of the invention, in addition to the aforementioned effects, unknown structures of the substance which is the inspection target for the multiple inspection types can also be determined with high accuracy.

A seventeenth aspect of the invention is a suspension for multiple inspection multiplexing according to any one of the twelfth through fourteenth aspects of the invention, wherein when inspecting for the presence or degree of presence of the inspection target of multiple inspection types, the labeled detection elements and bonding substances are substances selected so as to mutually bond only via the inspection target.

According to the seventeenth aspect of the invention, in addition to the above effects, the invention can be easily applied to inspections relating for example to whether or not a microbial species is present in a given sample. Accordingly, inspections for the presence of colon bacillus, O-157, or the like can be executed easily and accurately.

An eighteenth aspect of the invention is a suspension for multiple inspection multiplexing according to the fifteenth aspect of the invention, wherein when inspecting for the suitability of a structure of a predetermined inspection site of multiple inspection types for genetic material, the labeled detection element is a genetic material such as DNA fragments with each cleaved so that a single chain inspection site includes a single inspection type, and labeled, and the bonding substances for the inspection types are genetic material having a single chain base sequence selected so as to bond or not bond with the inspection sites of the genetic material, if the structure is normal or abnormal respectively.

According to the eighteenth aspect of the invention, in addition to the above effects, material cleaved so as to include a single strand inspection site on which inspection for the suitability of the structure of a single type of DNA or the like is performed, is used as the labeled detection element. Accordingly, it is possible to specify efficiently and accurately a plurality of mutations in the DNA base sequence, in parallel.

A nineteenth aspect of the invention is a suspension for multiple inspection multiplexing according to any one of the fifteenth through seventeenth aspects of the invention, wherein when inspecting the suitability of the structure for a predetermined inspection site for protein of multiple inspection types having predetermined immobilization sites, as to whether or not this exists or the degree of existence, the labeled detection element is the protein, and is labeled so that this is mutually identifiable for each of the multiple inspection types, by the labeling substance via a substance which is selected so as to specifically bond or not bond with the inspection site, and the bonding substance is a substance selected so as to specifically bond with the immobilization site. Here “substance” includes for example antibody.

According to the nineteenth aspect of the invention in addition to the above mentioned effect, for the labeled detection element there is one which is labeled with a labeling substance via an antibody group selected so as to bond or not bond with the inspection site, so as to perform inspection of the suitability of the structure for protein of one kind, as to as to whether or not this exists or the degree of existence. As a result, for the inspection site of the protein of multiple types, inspection for mutant forms of the protein can be performed in parallel, and quickly and efficiently.

A twentieth aspect of the invention is one where in the seventeenth aspect, with a genetic material having a predetermined base sequence of multiple inspection types, when inspecting for the presence or degree of presence in a DNA extraction liquid which suspends a known DNA, the labeled detection element group is a primer group of a known large number of multiple inspection types, which is multiply included for each of the inspection types, and labeled so as to be identifiable for each of the inspection types, and which for each of the inspection types begins synthesis and amplification for base sequences corresponding to each of the inspection types, and the bonding substance is a primer group of multiple inspection types paired with the primer group.

According to the twentieth aspect of the invention, in addition to the aforementioned effects, inspections to determine whether or not certain genetic material exists in a DNA extract in which unknown DNA which is the detection target of the multiple inspection types is suspended, can be preformed in parallel, and quickly and efficiently.

A twenty first aspect of the invention is a suspension for multiple inspection multiplexing according to the sixteenth aspect of the invention, wherein when inspecting to determine the base sequence of genetic material which has mutation sites where mutations are predicted, then regarding respective structure elements of the labeled detection elements there are primers having a base or a base sequence which is predicted to be mutated or inserted or not having a corresponding base or base sequence, in a position corresponding to the mutation site which is at the primer 3′end of the primer or the vicinity thereof, and structures different from this are labeled so as to be mutually identifiable, and regarding the bonding substance there are primers having a base or a base sequence which is predicted to be mutated or inserted or not having a corresponding base or base sequence, in a position corresponding to the mutation site which is at the 3′end of the primer or the vicinity thereof or separated upstream, and the fine particles carry a primer labeled for each of the structures via the primer.

According to the twenty first aspect of the invention, in addition to the aforementioned effects, unknown structures of the inspection target substance for the multiple inspection types can also be determined with high accuracy.

A twenty second aspect of the invention is a suspension for multiple inspection multiplexing according to any one of the twelfth through to twenty first aspects of the invention, wherein the fine particles can be remotely controlled by a magnetic field or the like.

According to the twenty second aspect of the invention, in addition to the aforementioned effects, by using fine particles such as magnetic particles which can to be remotely controlled, processing can be even more efficiently and easily carried out

A twenty-third aspect of the invention is one where in any one of the twelfth aspect, the fifteenth aspect and the seventeenth aspect, the labeled detection element is a DNA fragment with one end labeled and the other end having the cohesive end of an inspection site, and is obtained by providing upstream of a 3′ end, a type IIS restriction enzyme array with a double strand DNA having multiple inspection sites which are separated to the extent that bases in the recognition sites and the cleavage sites for each of the inspection sites do not overlap and do not exert an influence on the PCR primer, and processing this DNA by a type IIS restriction enzyme using labeled primers for multiple inspection types, and primers for multiple inspection types paired with these.

Here, “separated to the extent that bases in the recognition sites and the cleavage sites for each of the inspection sites do not overlap and do not exert an influence on the PCR primer” is preferably the case where separated by at least 10 bases. Ideally, separation to around twenty to thirty bases is preferable. However, with the presently known type IIS restriction enzyme, this is a maximum of around ten to twenty bases.

According to the twenty third aspect of the invention, in addition to the aforementioned effects, as the detection element, there is a DNA fragment with one end labeled and the other end having the cohesive end of an inspection site, and this is obtained by providing upstream of a 3′ end, a type IIS restriction enzyme array with a double strand DNA having multiple inspection sites in which the recognition sites and the cleavage sites are separated by at least 10 base pairs for each inspection site, and processing this DNA by a type IIS restriction enzyme using labeled primers for multiple inspection types, and primers for multiple inspection types paired with these.

Accordingly to this aspect of the invention, by using a type IIS restriction enzyme recognition sequence, it is possible to cleave the double strand DNA at any position without adversely affecting the inspection sites, which allows inspections which are diverse or versatile to be performed.

In the first through eleventh aspects, the fine particles are preferably ones which have for example magnetic particles, and which can be remotely controlled by a magnetic field or the like.

### BRIEF DESCRIPTION OF THE DRAWINGS

FIG. 1 is an explanatory drawing of a suspension and method according to a first embodiment of the present invention.

FIG. 2 is an explanatory drawing of a suspension and method according to a second embodiment of the present invention.

FIG. 3 is an explanatory drawing of a suspension and method according to a third embodiment of the present invention.

FIG. 4 is an explanatory drawing of a suspension and method according to a fourth embodiment of the present invention.

### BEST MODE FOR CARRYING OUT THE INVENTION

A multiple inspection multiplexing method and suspension for multiple inspection multiplexing according to embodiments of the present invention are described hereunder based on the drawings. It will be understood that these embodiments do not limit the present invention unless particularly specified.

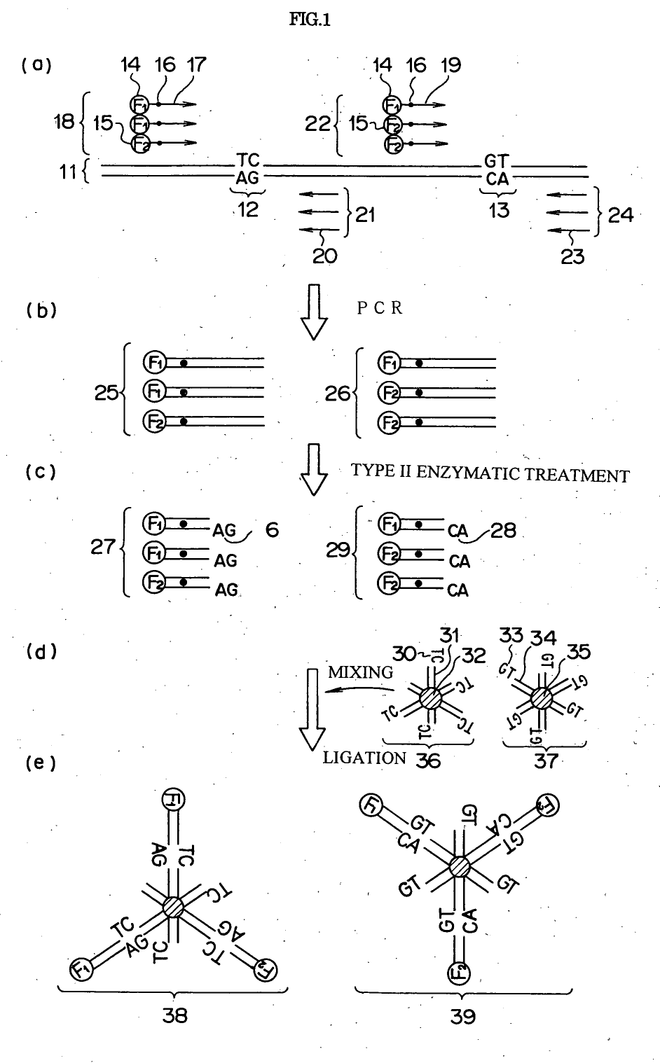

FIG. 1 shows a multiple inspection multiplexing method and a suspension for multiple inspection multiplexing according to a first embodiment.

As shown in FIG. 1 (a), this embodiment is one which performs an inspection of a structure of respective inspection objects having a base sequence being the inspection sites 12 and 13 of a plurality of places of specific double strand inspection DNA 11 being genetic material extracted from one patient, for example, to inspect as to whether or not the structure coincides with a predicted structure, that is, whether the structure is suitable.

The aforementioned “inspection type” here refers to types of inspection sites 12 and 13 where the positions on the inspection DNA 11 and the base sequence thereof are different. In this example, for simplicity of explanation, only the inspection sites 12 and 13 for two types is shown. Furthermore, the number of bases of the respective inspection sites 12 and 13 is only shown for the case of two bases, however this is not limited to this number of types and number of bases.

In FIG. 1 (a), furthermore, this has a primer group 18 comprising primers 17 containing type IIS restriction enzyme recognition sequences 16 with the bases in the recognition site and the cleavage site separated by at least 10 bases, with one end connected to either one of the labeling substances 14 and 15 of the two types, and provided on the upstream side of the 3′ end, so as to have a cleavage site on the inspection site 12.

Furthermore, this has a primer group 21 comprising primers 20 which have not been labeled paired with these primers 17. Here the aforementioned labeling substances 14 and 15 are for example fluorescence substances of two different types. For the fluorescence substance there is for example, an inducement substance such as FITC (fluorescein isothiocyanate), rhodamine, isothiocyanate, IRD40, Cy3 and the like, or an inorganic substance such as europium complex.

Also for the inspection site 13, similarly this has a primer group 22 comprising primers 19 containing the aforementioned type IIS restriction enzyme recognition sequence. Furthermore, this has a primer group 24 comprising primers 23 which have not been labeled paired with these primer 19.

Here in the primer group 18, the molar ratio of the primer 17 bonded with the labeling substance 14, and the primer 17 bonded with the labeling substance 15 (if the labeling substances in the primers are approximately evenly distributed, this approximately corresponds to the number ratio) is 2 to 1. On the other hand, in the primer group 22, the molar ratio of the primer 19 bonded with the labeling substance 14 and the primer 19 bonded with the labeling substance 15 is 1 to 2.

Next, in FIG. 1 (b), these inspection DNA 11, primer group 18 and primer group 21, and primer group 22 and primer group 24 are mixed, and PCR is executed.

As a result, as shown in FIG. 1 (b), the two types of double strand DNA fragment groups 25 and 26 which have the type IIS restriction enzyme recognition site in the vicinity of the labeled primer side end are synthesized in parallel.

Next, as shown in FIG. 1 (c), by processing the amplified DNA fragment groups 25 and 26 with the type IIS restriction enzyme, then a DNA fragment group 27 comprising multiple DNA fragments having labeling substances on the end, and having cohesive ends 6 of the inspection sites 12 on the other end, is generated, and similarly a DNA fragment group 29 comprising multiple DNA fragments having cohesive ends 28 of the inspection sites 13 on the other end, is generated. These DNA fragment groups 27 and 29 correspond to the labeled detection element group of the multiple inspection types.

The respective steps of FIG. 1 (a), (b), (c) described above correspond to the generating steps.

Next, as shown in FIG. 1 (d), fine particles 32 having DNA fragments 31 having cohesive ends 30 on the end, are prepared as the bonding substance. The cohesive ends 30 of the DNA fragments 31, in the case where the base sequence of the inspection site 12 is a normal type (in the case where the inspection site 12 is normal, this is GA), have complementary cohesive ends (TC).

The multiple collection of these, is a fine particle group 36 corresponding to the inspection site 12. Similarly fine particles 35 having DNA fragments 34 having cohesive ends 33 on the end, are prepared as the aforementioned bonding substance. The cohesive ends 33 of the DNA fragments 34, in the case where the base sequence of the inspection site 13 is a normal type (in the case where the inspection site 13 is normal, this is AC), have complementary cohesive ends (GT). The multiple collection of these, is a fine particle group 37 corresponding to the inspection site 13. The fine particles 32 and 35 each have only the same types of DNA fragments 31 and 34.

The DNA fragment groups 27 and 29 generated in FIG. 1 (c) and the fine particle groups 36 and 37 generated in FIG. 1 (d) are mixed, to perform a ligation reaction. As a result in the case where either of the inspection sites 12 and 13 is a normal type, the DNA fragments 31 and the DNA fragments 34 being the multiple bonding substance which each of the fine particle groups 36 and 37 have, bond with the multiple DNA fragment group 27 and the DNA fragment group 29 being the respective labeled detection elements. This FIG. 1 (d) corresponds to the processing step.

Since this bonding is conducted at random by the multiple substance members, the bondable bonding substances are bonded in a condition with the molar ratio of the labeling substance 14 and the labeling substance 15 of the detection elements excluding statistical error, maintained. This error becomes smaller the greater the number of items.

Consequently, as shown in FIG. 1 (e), the composite particle 38 and the composite particle 39 labeled under the condition where each of the labeling substance 14 and the labeling substance 15 are in different molar ratios for each of the inspection types, that is to say, either one of F1:F2=2:1, and F1:F2=1:2 or extremely close to this, are obtained.

On the other hand, in the case where either one of the inspection sites 12 and 13 is a mutation (a case where this is different from the array in d) then neither the composite particle 38 nor composite particle 39 can be detected.

Consequently, by conducting the aforementioned reaction in one reactor vessel, and detecting with a flow cytometer, it is possible to detect more than two types of mutation sites at the same time. Detection by means of this flow cytometer corresponds to the aforementioned detection step.

In the above description of the embodiment, it is only examined whether the structures of the inspection site 12 or the inspection site 13 (each AT, CA) are normal or abnormal. If the structures of each of the inspection site 12 and the inspection site 13 are both to be specified, this inspection can be performed by using the primer groups 18 which are formed so that the combinations of the base sequences corresponding to the structure of all the inspections sites which are pre-supposed are contained respectively in the 3′ end or in the vicinity thereof of the primer group 18, and which are labeled so that the primer group 18 labeled so as to be mutually identifiable, and are labelled so that all are mutually different for each of the respective inspection sites (12 and 13) as the structure elements of the labeled detection elements.

Furthermore, the two base cohesive ends which are used in the above description, can use for example cohesive ends of three or more bases such as four base cohesive ends according to the type IIS restriction enzyme of for example Fok 1 and the like. For example in the case of a four base cohesive end, the number of cases where recognition as different arrays is possible is 256 types, and the number of detection types comprising mutations of mutation sites which can be detected at the same time can be increased.

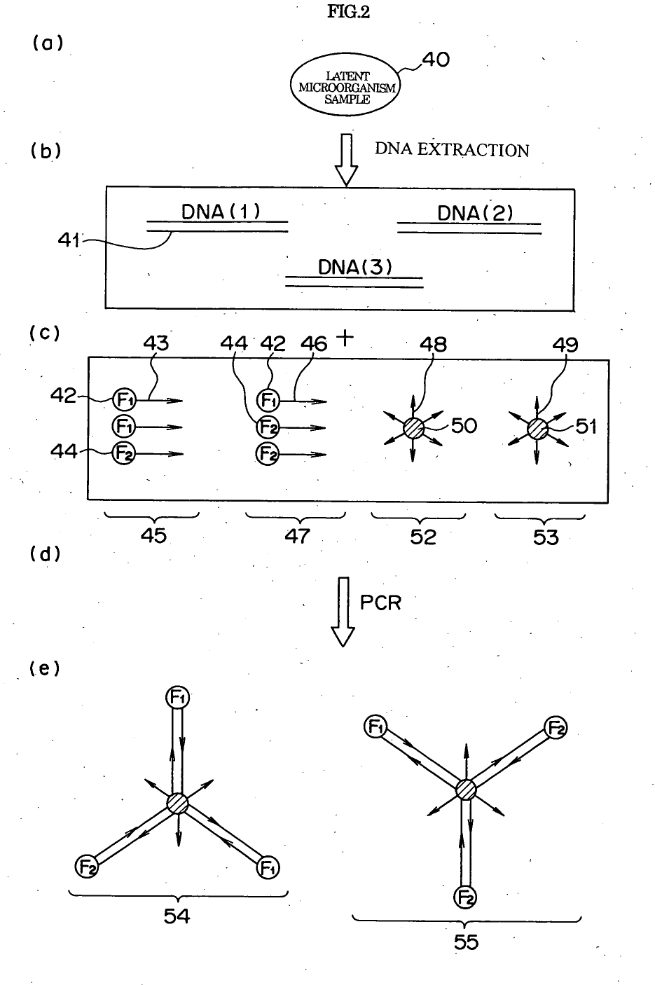

To continue, the multiple inspection multiplexing method and suspension for multiple inspection multiplexing according to a second embodiment is explained based on FIG. 2.

This example is one where the presence of a plurality of microbial species polluting foodstuffs or the like, is detected by reacting each one of the microbial species in a single reactor vessel.

In FIG. 2 (a), a latent microorganism sample 40 which is predicted to contain a plurality of microbial species is prepared. In FIG. 2 (b), various unknown types of inspection DNA (DNA (1), DNA (2), DNA (3), etc) 41 contained in this latent microorganism sample 40 are extracted from this latent microorganism sample 40.

Then, as shown in FIG. 2 (c), an inspection target which is the object of the inspection as to whether or not this is contained in the latent microorganism sample 40 is assumed to be for example a microbial species of two types (of course this may be a microbial species of three or more types). These two types correspond to the aforementioned inspection types.

Furthermore, primers 43 and 46 which are labeled (coded) by bonding the labeling substances of the two types such that these are each peculiar to the first microbial species and the second microbial species, are prepared. For these primers 43 and 46, ones with one end bonded with only one of a labeling substance (for example a fluorescence substance) 42 and 44 of the two types are multiply prepare, to give each of a primer group 45 and a primer group 47. These primer groups 45 and 47 correspond to the labeled detection element group of the labeled multiple inspection types.

However, in the primer group 45, the molar ratio of the primers 43 bonded with the labeling substances 42, and the primers 43 bonded with the labeling substances 44 (if the labeling substance in the primers are approximately evenly distributed, this approximately corresponds to the number ratio) is 2 to 1. On the other hand, in the primer group 47, the molar ratio of the primers 46 bonded with the labeling substances 42 and the primers 46 bonded with the labeling substances 44 is 1 to 2, being labeled so as to be mutually different. FIG. 2 (c) being the step up until performing this labeling, corresponds to the generation step.

As the bonding substance, one where primers 48 and 49 paired with the primers 43 and 46 are each immobilized on fine particles 50 and 51 is prepared. At this time, fine particle groups 52 and 53 of two inspection types comprising multiple fine particles 50 and 51 where only the primer 48 or the primer 49 having the same array are immobilized on the same fine particles 50 or fine particles 51 are prepared.

Next, the extracted DNA samples shown in FIG. 2 (b) are mixed as the inspection target in a suspension in which these primer group 45 and 47 and fine particle groups 52 and 53 are suspended, and PCR performed. As a result, in the case where the first microbial species and the second microbial species are not present in the sample, then according to the PCR method, by means of the primers 48 and 49 having the fine particles 50 and 51 and the labeled primers 43 and 46, double strand DNA fragments labeled as shown in FIG. 2 (d), are formed.

Formation of the double strands by the multiple primers 43 and 46 and the multiple primers 48 and 49 is carried out randomly. Therefore the double strands are formed in a condition where the molar ratio excluding statistical error of the labeling substances 42 of the labeled detection elements and the labeling substances 44 of the labeled detection elements is maintained.

This error becomes smaller the greater the number of items.

Consequently, as shown in FIG. 2 (d), the composite particles 54 and 55 labeled under the condition where each of the labeling substance 42 and the labeling substance 44 are in different molar ratios for each of the inspection types, that is to say, either one of F1:F2=2:1, and F1:F2=1:2 or extremely close to this, are obtained. Consequently, the presence of these composite particles 54 and 55 can be confirmed by detecting the molar ratio by analysis using a flow cytometer.

This FIG. 2 (d) and (e) corresponds to the processing step, and the analysis by means of the flow cytometer corresponds to the detection step.

On the other hand, in the case where no microbial species exist in the sample, then neither the composite particle 54 nor composite particle 55 are detected.

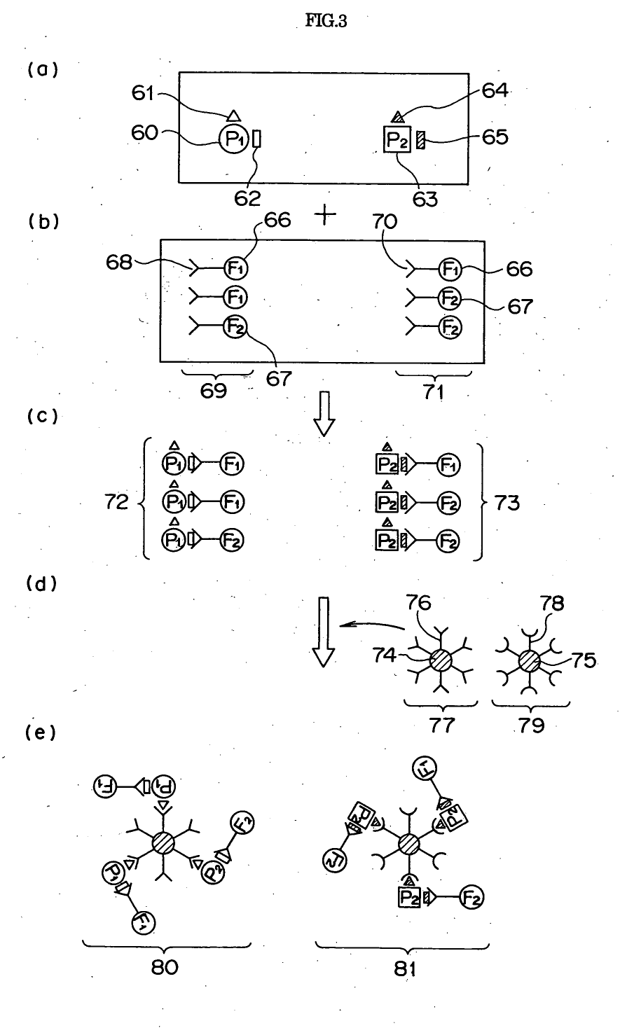

To continue, the suspension for multiple inspection multiplexing and the multiple inspection multiplexing method using this suspension according to a third embodiment is explained based on FIG. 3.

This embodiment is one where, for a plurality of different proteins present in a specimen, inspection as to whether or not there is a mutation of their respective structures is possible by reaction in a single container.

As shown in FIG. 3 (a), a rough extract containing protein (P1) 60 and a different type of protein (P2) 63 is prepared from a structure such as from humans.

The proteins 60 and 63 have predetermined immobilization sites 61 and 64 which are to be immobilized on later mentioned fine particle groups 77 and 79, and inspection sites 62 and 65 being sites which are the object of inspection for the suitability of a structure as to whether or not there is a mutation, whether or not this is present, or to what extent this is present.

As shown in FIG. 3 (b), in the case where the inspection sites 62 and 65 of the proteins 60 and 63 are a normal type, then this shows an antibody group 69 and 71 comprising labeled multiple antibodies 68 and 70 which specifically bond with the inspection sites 62 and 65.

In each antibody group 69, the ratio of the number of antibodies 68 bonded with the labeling substances 66 and the antibodies 68 bonded with the labeling substances 67 is 2 to 1. On the other hand, in the antibody group 71, the ratio of the number of antibodies 70 bonded with the labeling substances 66 and the antibodies 70 bonded with the labeling substances 67 is 1 to 2.

Next, these proteins 60 and 63 are introduced to a suspension in which the antibody groups 69 and 71 are suspended, and then reacted.

As a result, as shown in FIG. 3 (c), for the protein 60, a protein group 72 labeled such that the labeling substances 66 and 67 becomes 2 to 1 is obtained, and for the protein 63, a protein group 73 labeled such that the labeling substances 66 and 67 becomes 1 to 2 is obtained. These protein groups 72 and 73 correspond to the labeled detection element groups. That is to say, FIG. 3 (a), (b), (c) corresponds to the generation step.

On the other hand, as shown in FIG. 3 (d), fine particle groups 77 and 79 with multiple antibodies 76 and 78 which are bondable with the immobilization sites 61 and 64, immobilized on multiple fine particles 74 and 75, are prepared as the bonding substance. A proviso is that only the same antibodies are immobilized on the same particles.

Next, the labeled protein groups 72 and 73 are suspended in the suspension which suspends the fine particle groups 77 and 79 and mixed. In the case where the inspection sites 62 and 65 of the proteins 60 and 63 are normal type, then labeled composite particles 80 and 81 as shown in FIG. 3 (e) are formed.

Since mixing of the multiple antibodies 68 and 71 and the multiple proteins 60 and 63 is conducted at random, then the composite particles 80 and 81 are formed in a condition with the molar ratio of the labeling substance 66 of the detection element and the labeling substance 67 of the detection element excluding statistical error maintained. This error becomes smaller the greater the number of items.

Consequently, as shown in FIG. 3 (e), these are labeled under the condition where each of the labeling substance 66 and the labeling substance 67 are in different molar ratios for each of the proteins, that is to say, either one of F1:F2=2:1, and F1:F2=1:2 or extremely close to this. Consequently, the presence of these composite particles 80 and 81 can be confirmed by detecting the molar ratio for each one particle using a flow cytometer. This step of FIG. 3 (d) (e) corresponds to the processing step, and the detection by means of the flow cytometer corresponds to the detection step.

Furthermore, as another example of a suspension for multiple inspection multiplexing and a multiple inspection multiplexing method using this suspension according to the third embodiment, instead of the aforementioned antibodies 68 and 70, antibodies 68 and 70 which specifically bond with an inspection site for which the protein associated with the production of cancer has been mutated are selected, and whether or not this can be bonded due to the presence of the mutation is determined.

In this example, by measuring the fluorescence intensity of the respective composite particles 80 and 81 which have been bonded with this antibody group, the presence of mutated (in relation to cancer forming) protein, the difference or ratio of the quantity between the types of this protein, or the difference or ratio of the number of mutation sites per one protein, can be detected.

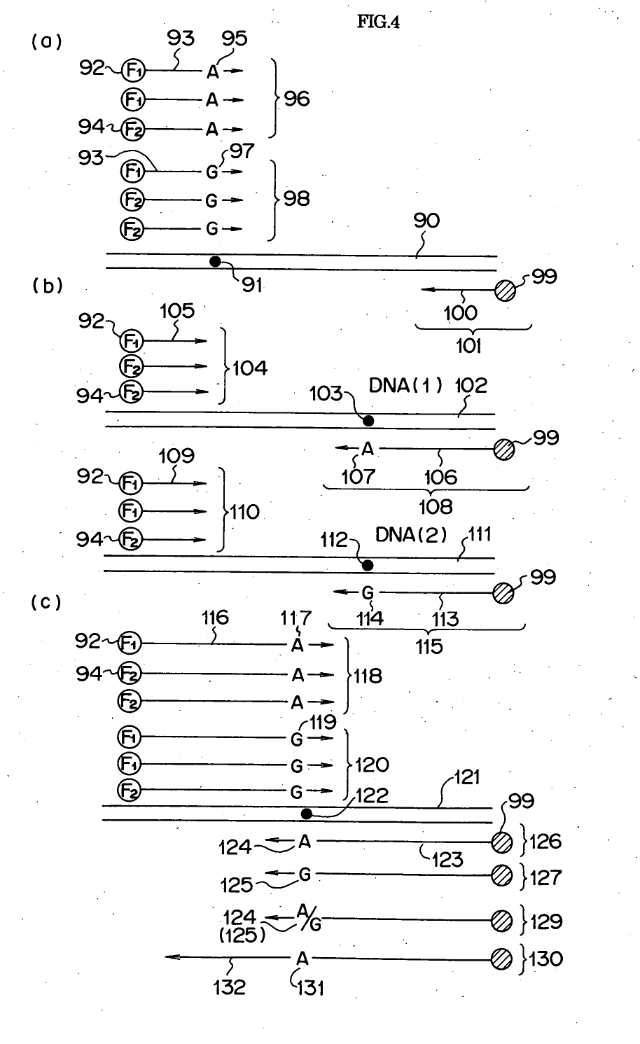

A suspension for multiple inspection multiplexing, and a multiple inspection multiplexing method using this suspension, according to a fourth embodiment is described based on FIG. 4.

This embodiment is one where for the second embodiment, in the case where a genetic material having a mutation site for which mutation is predicted is contained in genetic material having a predetermined base sequence of the multiple inspection types described in FIG. 2, for also specifying the structure of this mutation.

FIG. 4 (a) shows a method of determining the structure in the case where, for simplicity of explanation, a mutation site 91 of one base is present as an inspection site in the inspection DNA 90.

As the respective structure elements of the labeled detection elements, there is used suspended in a liquid, four-type primer groups 96 and 98 comprising multiple primers 93 each having one base A, G, T, C (in FIG. 4, for simplicity of explanation, only A and G is shown) for which a mutation is predicted in positions corresponding to the mutation site 91, at the 3′ end of the primers 93 or the vicinities 95 and 97 thereof.

The respective primers 93 belonging to the respective primer groups 96 and 98 having respective bases A, G (T, C) are bonded with only labeling substances 92 and 94 of one type, and the respective primer groups 96 and 98 bonded with the labeling substances 92 and 94 for each of the types, are a predetermined molar ratio; for example for the primer group 96, F1:F2=2:1 and for the primer group 98, F1:F2=1:2.

As the bonding substance, a primer 100 paired with the primer 93 is used. The multiple fine particles 99 having these multiple primers 100 constitute a fine particle group 101.

These primer groups 96 and 98, and the fine particle group 101 are suspended in liquid, and amplified by the PCR method, and by detecting complexes formed in the fine particles 99 bonded with either of the primer groups 96 and 98, the structure of the mutation site 91 can be specified.

FIG. 4 (b) is for determining, in the case where mutation sites 103 and 112 (for simplicity of explanation, this has a structure of one base) exist in each of the inspection DNA (1) 102, and inspection DNA (2) 111 of two types, the suitability of the structure of these mutation sites.

In this example, labeled primers 105 and 109 are prepared as the labeled detection elements. For the primers 105 and 109, ones where for example one end is bonded to only one of a labeling substance (for example a phosphorescence substance) of two types 92 and 94 are multiply prepared, to give each of a primer group 104 and a primer group 110.

In the primer group 104, the ratio of the number of items of; the primer 105 bonded with the labeling substance 92 and the primer 105 bonded with the labeling substance 94 is 1 to 2, and the ratio of the number of items of; the primer 109 bonded with the labeling substance 92 and the primer 105 bonded with the labeling substance 94 is 2 to 1, being labeled so as to be mutually different.

Furthermore, as the bonding substance, there is primers 106 and 113 each paired with the primers 105 and 109, and in the position corresponding to the mutation sites 103 and 112 of the 3′ ends or the vicinities thereof 107 and 117, fine particle groups 108 and 115 comprising fine particles 99 of two types having multiple primers 106 and 113 which each have a one base A, G predicted to mutate are prepared.

These primer groups 104 and 110, inspection DNA (1) 102 and DNA (2) 111, and fine particle groups 108 and 115 are suspended in liquid and amplified by the PCR method. As a result, the fluorescence having the molar ratio, is observed by the flow cytometer, for only the case where the mutation site 103 and 112 of the DNA (1) or DNA (2) has a relevant base, so that the suitability of the structure of the mutation position can be determined.

FIG. 4 (c) shows a method of determining the structure in the case where, for simplicity of explanation, a mutation site 122 of one base is present as an inspection site in the inspection DNA 121.

As the respective structure elements of the labeled detection elements, there is used four-type primer groups 118 and 120 comprising multiple primers 116 each having one base A, G, T, C (in FIG. 4, for simplicity of explanation, only A and G is shown) for which a mutation is predicted in positions 117 and 119 corresponding to the mutation site 122 at the 3′ end of the primer 116 or the vicinity 117 and 119 thereof. The primers 116 belonging to the respective primer groups 118 and 120 are bonded with only the labeling substances 92 and 94 of one type, and the primer groups 118 and 120 bonded with the labeling substances 92 and 94 for each of the respective types are in a predetermined molar ratio, for example in the primer group 118, F1:F2=2:1 and in the primer group 120 F1:F2=1:2.

Furthermore, as an example of the first fine particle group, in the positions 124 and 125 corresponding to the mutation site 122 of the 3′end or in the vicinity thereof paired with the primers 116, the multiple primers 123 having the bases A, G (T, C) which are predicted to mutate are used as the bonding substance. The fine particle groups 126 and 127 comprising the fine particles 99 of four types having these multiple primers 123 are used. By suspending these primer groups 118 and 120, the inspection DNA 121 and the fine particle groups 126 and 127 in a liquid and performing amplification by means of the PCR method, the fluorescence intensity ratio corresponding to the structure of the mutation site 122 is detected. Furthermore, by measuring the overall strength, it is possible to analyze in what proportion the DNA having the mutation site exists in the sample.

As an example of a second fine particle group, in the positions 124 and 125 corresponding to the mutation site 122 of the 3′end or in the vicinity thereof paired with the primers 116, the multiple primers 123 having the bases A, G (T, C) which are predicted to mutate are used as the bonding substance. The fine particle group 129 comprising the fine particles 99 of one type having these multiple primers 123 of four types so as to be the same molar ratio are used. By suspending these primer groups 118 and 120, the inspection DNA 121 and the fine particle group 129 in a liquid and performing amplification by means of the PCR method, the fluorescence intensity ratio corresponding to the mutation site 122 is detected. Furthermore, by measuring the overall strength, it is possible to analyze in what proportion the DNA having the mutation site exists in the sample.

As an example of a third fine particle group, in the positions 131 corresponding to the mutation site 131 which is at the interior separated from the 3′end paired with the primers 116, the multiple primers 132 of one base type having a suitable base, for example A (G, T, C also possible, or inosine) is used as the bonding substance. The fine particle group 130 comprising the fine particles 99 having these multiple primers 132 is used. In this example, since the position corresponding to the mutation site is separated from the 3′end, the base or the base array which comes to the position corresponding to the mutation site does not have a significant influence on the amplification by means of the PCR method, and hence a substance with a common bonding substance can be used. Consequently, in the case where the third fine particle group is used, then compared to the case of the first fine particle group, the inspection process can be simplified. By using the first fine particle group and the third fine particle group, parallel inspection in conditions where other DNA exists becomes possible.

By suspending these primer groups 118 and 120, the inspection DNA 121 and the fine particle group 130 in a liquid and performing amplification by means of the PCR method, the fluorescence intensity ratio corresponding to the structure of the mutation site 122 is detected. Furthermore, by measuring the overall strength, it is possible to analyze in what proportion the DNA having the mutation site exists in the sample.

The above embodiments are specifically described in order to better understand the present invention, but do not limit other forms. Consequently, modification is possible within a range which does not alter the gist of the invention. For example, in the above description, for convenience of explanation, the description is for where inspections of two inspection types and inspections using labeled detection elements of two types, are performed severally in parallel, and labeling is by labeling substances of two types. However inspection is not limited to this case and inspections of three or more inspection types, and inspections using labeling substances of three or more types may be conducted.

In the above example, the case where the labeling substance is a luminescent material is described, however the labeling substance is not limited to this example, and may be various substances having instantaneously quantifiable physical quantities such as magnetic field or nuclear spin condition. Furthermore, even with a luminescent material, not only the emission wavelength and the emission intensity, but also the degree of polarization of the emission, the emission phase, the emission life span and the like may be detected.

The above inspections are conducted for cases related to, polymorphism concerned with DNA, inspection of microorganism types, and mutation of protein, however the inspections are not limited to these examples, and needless to say these may also be used in inspections related to sugar or amino acid or the like.

Furthermore, also other than antibodies, lecitin, other proteins, low molecular substances, and the like, and substances specifically bonded to the inspection substance may be used. In this case, it is also possible to conduct inspections of various substances capable of specific bonding, comprising sugars, lipid and other low molecular weight and high molecular weight substances.

Moreover, in the above examples, the description was only for the case of mutations where the mutation site was one base or two bases. However, this is not limited to this example and needless to say this can be also applied for example to cases of mutations having base sequences comprising bases of three or more, and to cases where there is deficiency, and insertion. Furthermore, the method of analysis for the mutation site is not necessarily limited to the case of conducting parallel inspection of the multiple inspection types, and can also be used when conducting only inspection of one inspection type.

1. A method of multiple inspection multiplexing for conducting in parallel inspections of multiple inspection types comprising:
a generation step for generating labeled detection element groups for multiple inspection types labeled so as to be identifiable among respective inspection types;
a processing step in which at least a generated labeled detection element group for the multiple inspection types, and a fine particle group for the multiple inspection types having for each of the inspection types bonding substances for multiple inspection types selected according to the inspection description of the inspection types based on whether they bond with or do not bond with said labeled detection elements for each inspection type, are suspended in a liquid and processed, and
a detection step for detecting for each of said inspection types whether said labeled detection elements are carried by said fine particles or not or to what extent they are carried.

2. A method of multiple inspection multiplexing according to claim 1, wherein said generating step comprises a step for suspending and bonding in a liquid for each of the inspection types, multiple detection elements, and labeling substances for the inspection types with predetermined types included at a predetermined molar ratio, and said type or the molar ratio thereof are made different so as to be mutually identifiable for each of the inspection types.

3. A method of multiple inspection multiplexing according to claim 2, wherein said generating process comprises a step for distributing all of said labeling substances for each of the inspection types to approximately all of the labeled detection elements for each of the inspection types, and bonding a single said labeled detection element with only a labeling substance of one type.

4. A method of multiple inspection multiplexing according to any one of claim 1 through claim 3, wherein when inspecting for the suitability of the structure of an inspection target of the multiple inspection types, in said generating step said labeled detection elements are obtained by labeling the respective inspection targets, and in said inspecting step, said bonding substances are substances which are only bonded when said inspection target has a predetermined structure.

5. A method of multiple inspection multiplexing according to any one of claim 1 through claim 3, wherein when conducting an inspection to determine an unknown structure of an inspection target for each of the inspection types, in said generating step said labeled detection elements are known structure elements of multiple types expected to bond with said bonding substances, only when said unknown structure is present, and are labeled so as to be mutually different, and said unknown structure is determined from said known structure elements bonded to said bonding substance.

6. A method of multiple inspection multiplexing according to any one of claim 1 through claim 3, wherein when inspecting for the presence and degree of presence of the inspection target, said labeled detection elements and bonding substances in said generating step and said bonding step are substances selected so as to mutually bond only when said inspection target is present, and in said processing step, said inspection target is also suspended and processed.

7. A method of multiple inspection multiplexing according to any one of claim 1 through claim 3, wherein when inspecting for the suitability of a structure of a predetermined inspection site of multiple inspection types of genetic material, in said generating step genetic material, such as a DNA fragment, cleaved so that each single chain inspection site includes a single inspection type, and labeled, is generated as said labeled detection element, and said bonding substances for the inspection types are genetic material having a single chain base sequence selected so as to bond or not to bond with said inspection sites of said genetic material, if the structure is normal or abnormal.

8. A method of multiple inspection multiplexing according to claim 4, wherein said generating step comprises; an amplification step for amplifying a double strand DNA fragment by the PCR method by mixing: double strand DNA having inspection sites for multiple inspection types; a primer group labeled for each of the inspection types with a labeling substance bonded with one end, into which is inserted a recognition site of a type IIS restriction enzyme which has a cleavage site in which an inspection site downstream of a 3′end of the primer acts as a cohesive end; and a primer group paired with this primer group, and then amplifying double strand DNA fragments by the PCR method; and an enzyme reaction step for processing the amplified DNA fragments by a type IIS restriction enzyme, to generate as said labeled detection element, DNA fragments having the cohesive end of the inspection site at the other end, wherein
said processing step, for each inspection type, suspends in a liquid; said labeled detection element group, and a fine particle group of multiple inspection types having a DNA fragment which has a cohesive end having a base sequence capable of bonding when a base sequence of the cohesive end of said labeled detection element is normal, and mixes these, to perform a ligation reaction.

9. A method of multiple inspection multiplexing according to claim 6, wherein said generating step comprises an amplification step for suspending in a DNA extract in which multiple unknown DNA is suspended: a primer group labeled to as to be identifiable for each inspection type, and which for each of the inspection types begins DNA synthesis for known multiple inspection types which are the object of inspections; and fine particles which have many primer groups for multiple inspection types paired with said primer group, and amplifying by the PCR method.

10. A method of multiple inspection multiplexing according to claim 5, wherein when inspecting to determine the base sequence of genetic material which has mutation sites where mutations are predicted in each inspection type, in said amplification step,
as respective structure elements of said labeled detection elements there are primers having a base or a base sequence which is predicted to be mutated or inserted or not having a corresponding base or base sequence, in a position corresponding to said mutation site which is at said primer 3′end or the vicinity thereof, and structures different from this are labeled so as to be mutually identifiable,
and regarding said bonding substance there are fine particles having a large number of primers having a base or a base sequence which is predicted to be mutated or inserted or not having a corresponding base or base sequence, in a position corresponding to said mutation site which is at the 3′end of said primer or the vicinity thereof or separated upstream, and these are suspended in a DNA extract in which multiple unknown DNA is suspended and amplified by the PCR method.

11. A method of multiple inspection multiplexing according to any one of claim 1 through claim 5, wherein when inspecting the suitability of the structure for a predetermined inspection site for protein of multiple inspection types having predetermined immobilization sites, as to whether or not this exists or the degree of existence, said labeled detection element in said generation step and in said process step is a protein of multiple inspection types, and is labeled so that this is mutually identifiable for each of the multiple inspection types, by said labeling substance via a substance which is selected so as to bond or not bond with said inspection site, and said bonding substance is a substance selected so as to specifically bond with said immobilization site.

12. A suspension for multiple inspection multiplexing containing;
labeled detection element groups for multiple inspection types labeled so as to be identifiable among respective inspection types, and
a fine particle group for the multiple inspection types having for each of the inspection types bonding substances for multiple inspection types selected according to the inspection description of the inspection types based on whether they bond with or do not bond with said labeled detection elements for each inspection type,
and inspection of multiple inspection types is performed in parallel using said suspension by detecting for each of said inspection types whether said labeled detection elements are carried by said fine particles or not or to what extent they are carried.

13. A suspension for multiple inspection multiplexing according to claim 12, wherein all of said labeling substances for each of the inspection types which are labeled by only bonding said labeled detection elements for said respective inspection types with labeling substances of respective inspection types, are substances which contain predetermined types in predetermined molar ratios, and these types or the molar ratios thereof are made different so as to be mutually identifiable for each of the inspection types.

14. A suspension for multiple inspection multiplexing according to claim 13, wherein all of said labeling substances for each of the inspection types are distributed to approximately all of said labeled detection elements for each of the inspection types, and a single labeled detection element is bonded with only a labeling substance of one type.

15. A suspension for multiple inspection multiplexing according to any one of claim 12 through claim 14, wherein when inspecting for the suitability of the structure of an inspection target of the multiple inspection types, said labeled detection element groups are said inspection target groups labeled so as to be different for each inspection type thereof, and said bonding substance groups are substances which are only bonded when the labeled inspection target group thereof has a predetermined structure.

16. A suspension for multiple inspection multiplexing according to any one of claim 12 through claim 14, wherein when conducting an inspection to determine an unknown structure of an inspection target for each of the inspection types, said labeled detection elements are known structure elements of multiple types labeled so as to be mutually different and expected to bond with said bonding substances, only when said unknown structure is present, and said unknown structure is determined from said known structure elements bonded to said bonding substance.

17. A suspension for multiple inspection multiplexing according to any one of claim 12 through claim 14, wherein when inspecting for the presence or degree of presence of the inspection target of multiple inspection types, said labeled detection elements and bonding substances are substances selected so as to mutually bond only via said inspection target.

18. A suspension for multiple inspection multiplexing according to claim 15, wherein when inspecting for the suitability of a structure of a predetermined inspection site of multiple inspection types for genetic material, said labeled detection element is a genetic material such as DNA fragments with each cleaved so that a single chain inspection site includes a single inspection type, and labeled, and said bonding substances for the inspection types are genetic material having a single chain base sequence selected so as to bond or not bond with said inspection sites of said genetic material, if the structure is normal or abnormal.

19. A suspension for multiple inspection multiplexing according to any one of claim 15 through claim 17, wherein when inspecting the suitability of the structure for a predetermined inspection site for protein of multiple inspection types having predetermined immobilization sites, as to whether or not this exists or the degree of existence, said labeled detection element is said protein, and is labeled so that this is mutually identifiable for each of the multiple inspection types, by said labeling substance via a substance which is selected so as to specifically bond or not bond with said inspection site, and said bonding substance is a substance selected so as to specifically bond with said immobilization site.

20. A suspension for multiple inspection multiplexing according to claim 17, wherein, with a genetic material having a predetermined base sequence of multiple inspection types, when inspecting for the presence or degree of presence in a DNA extraction liquid which suspends a known DNA, said labeled detection element group is a primer group of a known large number of multiple inspection types, which is multiply included for each of the inspection types, and labeled so as to be identifiable for each of the inspection types, and which for each of the inspection types begins synthesis and amplification for base sequences corresponding to each of the inspection types, and said bonding substance is a primer group of multiple inspection types paired with said primer group.

21. A suspension for multiple inspection multiplexing according to claim 16, wherein when inspecting to determine the base sequence of genetic material which has mutation sites where mutations are predicted, then regarding respective structure elements of said labeled detection elements there are primers having a base or a base sequence which is predicted to be mutated or inserted or not having a corresponding base or base sequence, in a position corresponding to said mutation site which is at the primer 3′end of said primer or the vicinity thereof, and structures different from this are labeled so as to be mutually identifiable, and regarding said bonding substance there are primers having a base or a base sequence which is predicted to be mutated or inserted or not having a corresponding base or base sequence, in a position corresponding to said mutation site which is at the 3′end of said primer or the vicinity thereof or separated upstream, and said fine particles carry a primer labeled for each of said structures via said primer.

22. A suspension for multiple inspection multiplexing according to any one of claim 12 through to claim 21, wherein said fine particles can be remotely controlled by a magnetic field or the like.

23. A suspension for multiple inspection multiplexing according to any one of claim 12, claim 15 and claim 17, wherein said labeled detection element is a DNA fragment with one end labeled and the other end having the cohesive end of an inspection site, and is obtained by providing upstream of a 3′ end, a type IIS restriction enzyme array with a double strand DNA having multiple inspection sites which are separated to the extent that bases in the recognition sites and the cleavage sites for each of the inspection sites do not overlap and do not exert an influence on the PCR primer, and processing this DNA by a type IIS restriction enzyme using labeled primers for multiple inspection types, and primers for multiple inspection types paired with these.
